(19) United States
(12) Patent Application Publication
(10) Pub. No.: US 2007/0092493 A1
(43) Pub. Date: Apr. 26, 2007

(54) Living cell sheet

(76) Inventors: Hsing-Wen Sung, Hsinchu (TW); Chun-Hung Chen, Hsinchu (TW); Hosheng Tu, Newport Beach, CA (US)

Correspondence Address: HOSHENG TU, 15 RIEZ, NEWPORT BEACH, CA, 92657-0116, US

(21) Appl. No.: 11/256,729

(22) Filed: Oct. 24, 2005

(51) Int. Cl. A61K 35/12; C12N 5/08

(52) U.S. Cl. 424/93.7; 435/366

(57) ABSTRACT

A novel method, using a thermoreversible MC/PBS/Collagen hydrogel coated on the TCPS dish, for harvesting a living cell sheet with ECM. The coated hydrogel system is reusable and can be used for culturing a multi-layer cell sheet. The obtained living cell sheets may be used for tissue reconstructions.

### FIELD OF THE INVENTION

The present invention is related to living cell sheets for tissue reconstructions and regeneration, more particularly, the invention is related to sheets derived from a thermoreversible hydrogel coated on a tissue culture polystyrene dish for harvesting living cells.

### BACKGROUND OF THE INVENTION

Methylcellulose (MC) is a water-soluble polymer derived from cellulose, the most abundant polymer in nature. As a viscosity-enhancing polymer, it thickens a solution without precipitation over a wide pH range. This feature makes it widely useable as a thickener in the food and paint industries. It is recognized as an acceptable food additive by the U.S. Food and Drug Administration. Additionally, the physiological inertness and the storage stability of MC permit its use in cosmetics and pharmaceutical products.

Recently, investigations of hydrogels have focused on functional hydrogels. These functional hydrogels may change their structures as they expose to varying environment, such as temperature, pH, or pressure. MC becomes gels from aqueous solutions upon heating or salt addition (Langmuir 2002;18:7291, Langmuir 2004;20:6134). This unique phase-transition behavior of MC makes it as a promising functional hydrogel for various biomedical applications (Biomaterials 2001;22:1113, Biomacromolecules 2004;5:1917). Tate et al. studied the use of MC as a thermoresponsive scaffolding material (Biomaterials 2001;22:1113). In their study, MC solutions were produced to reveal a low viscosity at room temperature and formed a soft gel at 37° C.; thus making MC well suited as an injectable scaffold for the repair of defects in the brain. Additionally, using its thermoresponsive feature, MC was used by our group to harden aqueous alginate as a pH-sensitive based system for the delivery of protein drugs (Biomacromolecules 2004;5:1917).

It is disclosed herein that a novel application of this thermoresponsive MC hydrogel is blended with distinct salts and coated on tissue culture polystyrene (TCPS) dishes as a living-cell-sheet harvest system. It was reported that a thermoresponsive polymer, poly(N-isopropylacrylamide) (PNIPAAm), is chemically grafted on TCPS dishes to develop a cell-sheet for tissue reconstructions (J. Biomed. Mater. Res. 1993;27:1243). PNIPAAm is hydrophobic at 37° C. and hydrophilic at 20° C., thus the cultured cells can be harvested as a continuous cell sheet after incubation at 20° C. The harvested cell sheets have been used for various tissue reconstructions, including ocular surfaces, periodontal ligaments, cardiac patches, and bladder augmentations (Materials today 2004;42). In their method, PNIPAAm is polymerized and concurrently grafted to TCPS dishes by means of irradiation with an electron beam. The whole grafting process is relatively complicated and time-consuming (Tissue Eng. 2005;11:30).

It is herein disclosed that a simple and inexpensive method is provided by simply pouring aqueous MC solutions blended with distinct salts on TCPS dishes at room temperature (about 20° C.) and subsequently gelled at 37° C. (the MC hydrogel). The gelled coating at 37° C. is then evenly spread with a neutral aqueous collagen at 4° C. The spread aqueous collagen gradually reconstitutes with time and thus forms a thin layer of collagen coated on the MC hydrogel. The physical behaviors of the prepared MC hydrogels transition from the solution to gel states as a function of temperature.

There provides, therefore, a novel method, using a thermoreversible MC/PBS/Collagen hydrogel coated on the TCPS dish, for harvesting a living cell sheet with ECM. The coated hydrogel system is reusable and can be used for culturing a multi-layer cell sheet. The obtained living cell sheets are useful for tissue reconstructions and cell separation.

### SUMMARY OF THE INVENTION

Some aspects of the invention relate to a novel yet simple method, using a thermoreversible hydrogel system that is coated on tissue culture polystyrene (TCPS) dishes, for providing harvesting living cell sheets. The hydrogel system is prepared by simply pouring aqueous methylcellulose (MC) solutions blended with distinct salts on TCPS dishes at 20° C. In one embodiment, aqueous MC compositions form a gel at 37° C. for the applications of cell culture. In one embodiment, the hydrogel coating composed of 8% MC blended with 10 g/L PBS (the MC/PBS hydrogel, with a gelation temperature of about 25° C.) stayed intact throughout the entire course of cell culture.

Some aspects of the invention relate to cell attachments comprising evenly spreading the MC/PBS hydrogel at 37° C. with a neutral aqueous collagen at 4° C. The spread aqueous collagen gradually reconstitutes with time and thus forms a thin layer of collagen (the MC/PBS/Collagen hydrogel). After cells reaching confluence, a continuous monolayer cell sheet forms on the surface of the MC/PBS/Collagen hydrogel. When the grown cell sheet is placed outside of the incubator at 20° C., it detaches gradually from the surface of the thermoreversible hydrogel spontaneously, in absence of any enzymes.

Some aspects of the invention relate to a method of preparing a living cell sheet comprising: coating a thermoreversible hydrogel on a tissue culture dish, wherein the hydrogel comprises methylcellulose, phosphate buffered saline, and optionally collagen; loading target living cells into the dish; incubating the dish for a predetermined duration; and removing the sheet from the dish.

Some aspects of the invention relate to a method of preparing a 3-D living cell construct comprising: coating a thermoreversible hydrogel on a 3-D scaffold support element, wherein said hydrogel comprises methylcellulose, phosphate buffered saline, and collagen; loading target living cells onto said support element; and incubating said support element for a predetermined duration. In one embodiment, the method further comprises a step of removing said construct from said support element.

The results obtained in the MTT assay demonstrate that the cells cultured on the surface of the MC/PBS/Collagen hydrogel had better cell activities than those cultured on an uncoated TCPS dish. After harvesting the detached cell sheet, the remained viscous hydrogel system is reusable. Additionally, the developed hydrogel system is used for culturing a multi-layer cell sheet. The obtained living cell sheets are candidates for tissue reconstructions or tissue regeneration. In one embodiment, the cells of the invention comprise mesenchymal stem cells and adult multipotent cells.

### BRIEF DESCRIPTION OF THE DRAWINGS

FIG. 1 shows the DSC thermograms of aqueous methylcellulose solutions (2% by w/v) blended with distinct concentrations of NaCl.

FIG. 2 shows gelation temperatures of aqueous methylcellulose solutions blended with distinct salts: effect of the concentration of salt.

FIG. 3 shows gelation temperatures of aqueous methylcellulose solutions blended with distinct salts: effect of the concentration of methylcellulose.

FIG. 4 shows osmolalities of aqueous methylcellulose solutions blended with distinct salts: effect of the concentration of salt.

FIG. 5 shows osmolalities of aqueous methylcellulose solutions blended with distinct salts: effect of the concentration of methylcellulose.

FIG. 6 shows changes in osmolality of the PBS solution loaded on each studied TCPS dish with time.

FIG. 7 shows photographs of the TCPS dish coated with the MC/PBS hydrogel in sequence: (a) at 20° C.; (b) at 37° C. for 5 min; (c) at 37° C. for 30 min; (d) followed by at 20° C. for 2 min; (e) followed by at 20° C. for 20 min.

FIG. 8 shows photomicrographs of cells cultured on: (a) an uncoated TCPS dish, 40×; (b) the TCPS dish coated with the 2% MC+1M NaCl hydrogel, 40×; (c) the TCPS dish coated with the 2% MC+0.2 M Na₂SO₄ hydrogel, 40×; (d) the TCPS dish coated with the 2% MC+0.2M Na₃PO₄ hydrogel, 40×; and (e) the TCPS dish coated with the MC/PBS (8% MC+10 g/L PBS) hydrogel, 40× and (f) 100×.

FIG. 9 shows schematic illustrations of cells cultured on the TCPS dish coated with the MC/PBS/Collagen hydrogel and detachment of its grown cell sheet.

FIG. 10 shows photomicrographs of cells cultured on: (a) an uncoated TCPS dish; and (b) on the TCPS dish coated with the MC/PBS/Collagen hydrogel for 1, 3, and 7 days, respectively.

FIG. 11 shows photographs of (a) a grown cell sheet on the TCPS dish coated with the MC/PBS/Collagen hydrogel, and (b) its detaching cell sheet. Photomicrographs of the detaching cell sheet with time as (c) to 0).

FIG. 12 shows immunofluorescence images of the cell sheets grown on the TCPS dish coated with the MC/PBS/Collagen hydrogel for: (a) 1 week; and (b) 2 weeks.

FIG. 13 shows immunofluorescence images of: (a) a single-layer cell sheet (CS); (b) a double-layer cell sheet; and (c) a tri-layer cell sheet obtained from the TCPS dish coated with the MC/PBS/Collagen hydrogel; and (d) a tri-layer cell sheet obtained by folding a single-layer cell sheet.

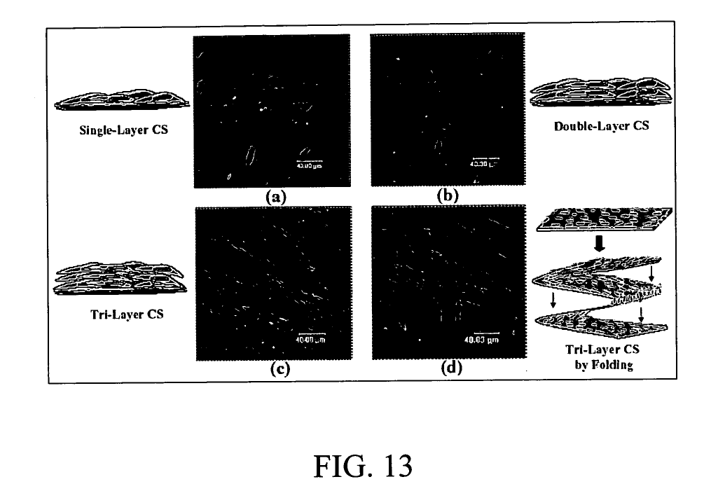

### DETAILED DESCRIPTION OF THE EXEMPLARY EMBODIMENTS

The preferred embodiments of the present invention described below relate particularly to preparation of sheets derived from a thermoreversible hydrogel coated on a tissue culture polystyrene dish for harvesting living cells. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

### Example No. 1 Gelation of Aqueous MC Solutions

Commercial MC is a heterogeneous polymer consisting of highly substituted zones (hydrophobic zones) and less substituted ones (hydrophilic zones). Aqueous MC solutions undergo a sol-gel reversible transition upon heating or cooling. In the solution state at lower temperatures, MC molecules are hydrated and there is little polymer-polymer interaction other than simple entanglements. As temperature is increased, aqueous MC solutions absorb energy (the endothermic peaks observed in the differential scanning calorimeter, DSC, thermograms discussed later) and gradually lose their water of hydration. Eventually, a polymer-polymer association takes place, due to hydrophobic interactions, causing cloudiness in solution and subsequently forming an infinite gel-network structure (Carbohydr. Polym. 1995;27:177).

The temperature in forming this gel-network structure, at which the aqueous MC solution does not flow upon inversion of its container, is defined as the gelation temperature herein. Therefore, the gelation temperature of the aqueous MC solution determined by inverting its container should be slightly greater than the onset temperature of the endothermic peak observed in its corresponding DSC thermogram.

It was reported that addition of salts lowers the gelation temperature of the aqueous MC solution (Langmuir 2002;18:7291). Upon addition of salts, water molecules are placed themselves around the salts, thus reducing the intermolecular hydrogen-bond formations between water molecules and the hydroxyl groups of MC. This can increase the hydrophobic interaction between MC molecules and lead to a decrease in their gelation temperature.

### Example No. 2 Preparation of Aqueous MC Solutions

MC (with a viscosity of 3,000-5,500 cps for a 2% by w/v aqueous solution at 20° C.) was obtained from Fluka (64630 Methocel® MC, Buchs, Switzerland). Aqueous MC solutions in different concentrations (1%, 2%, 3%, or 4% by w/v) were prepared by dispersing the weighed MC powders in heated water with the addition of distinct salts (NaCl, Na₂SO₄, Na₃PO₄) or in phosphate buffered saline (PBS) in varying concentrations at 50° C. The osmolalities of the prepared aqueous MC solutions were then measured using an osmometer (Model 3300, Advanced Instruments, Inc., Norwood, Mass., USA).

### Example No. 3 Gelation Temperatures of Aqueous MC Solutions

The physical gelation phenomena of aqueous MC solutions with temperature were visually observed and measured by a DSC (Pyris Diamond, Perkin Elmer, Shelton, Conn., USA). Aqueous MC solutions blended with distinct salts (2 ml samples) were exposed to elevating temperatures via a standard hot-water bath. Behavior was recorded at intervals of approximately 0.5° C. over the range of 20-70° C. The heating rate between measurements was approximately 0.5° C./min. At each temperature interval, the solutions/gels were allowed to equilibrate for 30 min. A “gel” criterion was defined as the temperature at which the solution did not flow upon inversion of the container. A DSC was used to determine the transition temperatures of the prepared aqueous MC solutions heating from 20 to 90° C. A heating rate of 10° C./min was used for all test samples.

### Example No. 4 Preparation of the MC-Hydrogel Coated TCPS Dish

The prepared aqueous MC solutions that had a gelation temperature below 37° C. were used to coat TCPS dishes (Falcon® 3653, diameter 35 mm, Becton Dickinson Labware, Franklin Lakes, N.J., USA). A 450 μl of test MC solutions was poured into the center of each TCPS dish at room temperature (about 20° C.). A thin transparent layer of the poured solution was evenly distributed on the TCPS dish. Subsequently, the TCPS dish was pre-incubated at 37° C. for 1 hour and a gelled opaque layer (the MC hydrogel) was formed on the dish. To evaluate whether the salts blended in the MC hydrogel would leach out with time, the coated TCPS dish was loaded with a pre-warmed PBS at 37° C. (2 ml, with an osmolality of 280±10 mOsm/kg). The osmolality of the loaded PBS solution was monitored with time. An uncoated TCPS dish loaded with the same PBS was used as a control.

For the system further coated with collagen, a 0.5 mg/ml aqueous type I collagen (bovine dermis collagen, Sigma Chemical Co., St. Louis, Mo., USA), adjusted to pH 7.4 by dialysis against PBS at 4° C., was evenly spread onto the aforementioned TCPS dish coated with the MC hydrogel at 37° C.

### Example No. 5 Cell Culture

HFF (human foreskin fibroblasts) were cultured in Dulbecco's modified Eagle's Minimal Essential Medium (12800 Gibco, Grand Island, N.Y., USA) supplemented with 10% fetal bovine serum (JRH, Brooklyn, Australia) and 0.25% penicillin-streptomycin (15070 Gibco, Grand Island, N.Y., USA) in the TCPS dish of Example No. 4. The cells were maintained at 37° C. with 5% CO₂ and the cultured media were changed 3 times a week until ready for use. In one embodiment, some appropriate growth factors may be added into the culture media, wherein the growth factor may be selected from the group consisting of VEGF (vascular endothelial growth factor), VEGF 2, bFGF (basic fibroblast growth factor), aFGF (acidic fibroblast growth factor), VEGF121, VEGF165, VEGF189, VEGF206, PDGF (platelet derived growth factor), PDAF (platelet derived angiogenesis factor), TGF-β (transforming growth factor-β1, β2, β3 and the like), PDEGF (platelet derived epithelial growth factor), PDWHF (platelet derived wound healing factor), insulin-like growth factor, epidermal growth factor, hepatocytic growth factor, and combinations thereof. After reaching confluence, cells were isolated from culture dishes with a 0.05% trypsin and then seeded uniformly on the coated TCPS dishes at a density of 4×10⁴ cells/cm² at 37° C. Cell attachment and growth were observed daily using a microscope. An uncoated TCPS dish was used as a control. Cell viability was assessed by the MTT [3-(4,5-dimethylthiazol-yl)-2,5-diphenyltetrazolium bromide, Sigma] assay. Details of the methodology used in the MTT assay were previously described (J. Biomed. Mater. Res.2002;61:360).

### Example No. 6 Detachment of Cell Sheets

Cells grown on the dishes for 1 or 2 weeks (with media changes 3 times per week) were taken out from the incubator with media present. The dishes were then allowed to cool at approximately 20° C. Changes in morphology of cell sheets on the dishes with time were photographed every 5 seconds for up to 15 min.

### Example No. 7 Immunofluorescence Staining

Monoclonal mouse anti-collagen type I (1:150, ICN Biomedicals, Inc., Aurora, Ohio, USA) and type III (1:200, Chemicon International Inc., Temecula, Calif., USA) antibodies were used for localizing type I and type III collagen secreted by HFF, respectively. A Cy5-conjugated affinity-purified goat anti-mouse IgG+IgM (H+L) (1.5 mg/ml, Jackson ImmunoResearch Laboratories, Inc., PA, USA) was used as the secondary antibody for labeling the monoclonal antibody. Cell sheets grown on the dishes were fixed in 4% phosphate buffered formaldehyde at 37° C. for 10 minutes and then permeabilized with 0.1% Triton X-100 in PBS containing 1% bovine serum albumin (PBS-BSA) and RNase 100 μg/ml. After washing 3 times with PBS-BSA, the cell sheets were exposed to the primary antibody for 60 min at 37° C. The cell sheets were then incubated for another 60 min with the secondary antibody (1:400) at room temperature. Additionally, the cell sheets were co-stained to visualize F-actins and nuclei acids by phalloidin (Oregon Greene® 514 phalloidin, Molecular Probes, Inc., Eugene, Oreg., USA) and propidium iodide (PI, P4864, Sigma), respectively.

Subsequently, the stained cell sheets were evenly mounted on the slides and examined with excitations at 488, 543, and 633 nm, respectively, using an inversed confocal laser scanning microscope (TCS SL, Leica, Germany). Superimposed images were performed with an LCS Lite software (version 2.0).

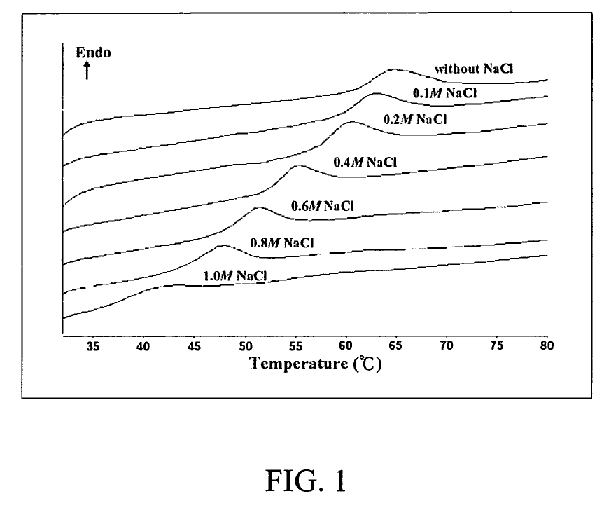

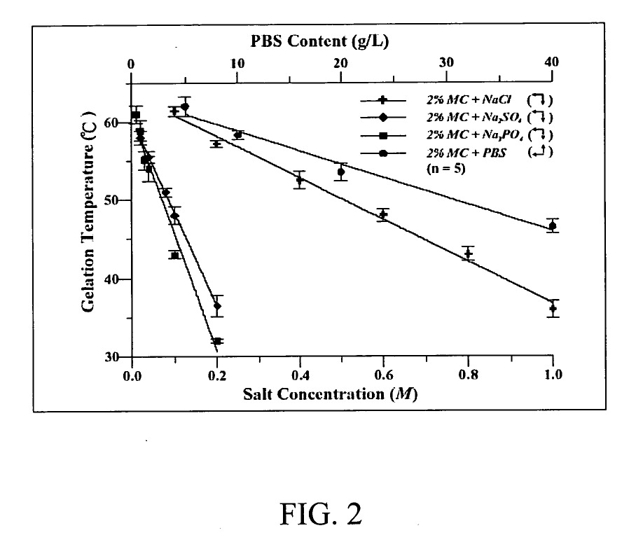

The salts blended in aqueous MC solutions played an important role in their physical sol-gel behavior. Examples of the DSC thermograms of aqueous MC solutions (2% by w/v) blended with distinct concentrations of NaCl are shown in FIG. 1. An endothermic peak was observed for each test sample in the heating process. With increasing the concentration of NaCl, the endothermic peak shifted to the left (p<0.05). This indicates that addition of NaCl in the aqueous MC solution led to its sol-gel transition occur at a lower temperature. Additionally, a higher concentration of NaCl used, a lower temperature in its sol-gel transition was observed. This fact was also observed in the determination of the gelation temperature of each test sample by inverting its container (FIG. 2). As expected, the onset temperatures of the endothermic peaks of aqueous MC solutions observed in the DSC thermograms were lower than their corresponding gelation temperatures obtained by the inversion method, ranged approximately from 1° C. to 3° C. (Table 1).

TABLE 1
The onset temperatures (Tₒₙₛₑₜ) of the endothermic peaks
of aqueous methylcellulose solutions (2% by w/v) blended with
distinct salts in varying concentrations (Conc.) observed in the
DSC thermograms and their gelation temperatures
(T_{gelation}) measured by an inversion method (n = 5).
NaCl

| | Conc. (M) | | | | | |
|---|---|---|---|---|---|---|
| | 0.1 | 0.2 | 0.4 | 0.6 | 0.8 | 1.0 |
| Tₒₙₛₑₜ | 59.0 ± | 55.6 ± | 52.0 ± | 47.4 ± 0.3 | 42.3 ± 0.4 | 35.2 ± 0.3 |
| | 0.8 | 0.3 | 0.1 | | | |
| T_{gelation} | 61.4 ± | 57.2 ± | 52.5 ± | 48.0 ± 0.8 | 43.0 ± 0.9 | 36.0 ± 1.1 |
| | 0.6 | 0.4 | 1.1 | | | |

Na₂SO₄

| | Conc. (M) | | | | |
|---|---|---|---|---|---|
| | 0.02 | 0.04 | 0.08 | 0.10 | 0.20 |
| Tₒₙₛₑₜ | 57.3 ± 0.2 | 54.8 ± 0.3 | 50.4 ± 0.4 | 47.4 ± 0.3 | 35.1 ± 0.3 |
| T_{gelation} | 58.0 ± 0.8 | 55.5 ± 0.7 | 51.0 ± 0.5 | 48.0 ± 1.1 | 36.5 ± 1.3 |

Na₃PO₄

| | Conc. (M) | | | | | |
|---|---|---|---|---|---|---|
| | 0.01 | 0.02 | 0.03 | 0.04 | 0.10 | 0.20 |
| Tₒₙₛₑₜ | 60.1 ± | 58.4 ± | 54.6 ± | 53.4 ± 0.3 | 42 ± 0.4 | 30 ± 0.2 |
| | 0.5 | 0.5 | 0.5 | | | |
| T_{gelation} | 61.0 ± | 58.9 ± | 55.1 ± | 54.0 ± 1.7 | 43 ± 1.1 | 32 ± 1.3 |
| | 1.1 | 1.3 | 1.1 | | | |

PBS

Conc. (g/L)

| | 5 | 10 | 20 | 30 |
|---|---|---|---|---|
| Tₒₙₛₑₜ | 57.5 ± 0.2 | 55.1 ± 0.5 | 52.4 ± 0.3 | 44.1 ± 0.2 |
| T_{gelation} | 62.0 ± 1.2 | 58.3 ± 0.5 | 53.5 ± 1.1 | 46.5 ± 0.9 |

Similar phenomena were observed when Na₂SO₄, Na₃PO₄, or PBS was blended into aqueous MC solutions (FIG. 2 and Table 1). Normally, an electrolyte (the salt blended) has a greater affinity for water than polymers resulting in removing water of hydration from the polymer and thus dehydrating or ‘salting out’ the polymer. The ability of an electrolyte to salt out a polymer from its solution generally follows the salt order in the lyotropic series. The cations follow the order Li⁺>Na⁺>K⁺>Mg²⁺>Ca²⁺>Ba²⁺, and more common anions follow the order PO₄³⁻>SO₄²⁻>tartrate>Cl⁻>NO₃⁻>Br⁻>I⁻>SCN⁻ (Int. J. Pharm. 1990;99:233). Accordingly, more water molecules were removed from aqueous MC solutions when Na₂SO₄ or Na₃PO₄ was added in the polymeric hydrogel, resulting in a lower gelation temperature. As shown in FIG. 2 and Table 1, at the same concentration of the salt blended, generally, the gelation temperatures of aqueous MC solutions followed the order Na₃PO₄<Na₂SO₄<NaCl(p<0.05).

Effects of addition of PBS in aqueous MC solutions on the onset temperatures of the endothermic peaks observed in the DSC thermograms and their gelation temperatures were similar to those blended with NaCl, Na₂SO₄, or Na₃PO₄ (FIG. 2 and Table 1). It was reported that the effect of cations on salting-out polymers in solution is less significant than that of anions. Therefore, salting-out MC polymers from aqueous solutions blended with PBS was mainly caused by its constituent anions such as Cl⁻, HPO₄²⁻, or H₂PO⁴⁻.

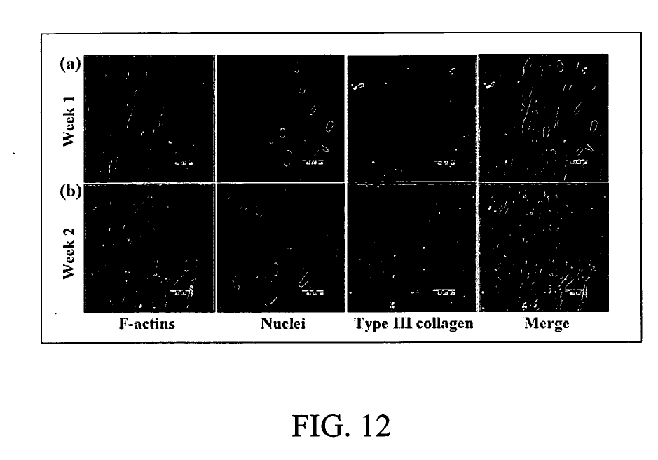

Results of the immunofluorescence images of the cell sheets grown on the MC/PBS/Collagen hydrogel for 1 and 2 weeks are shown in FIGS. 12a and 12b, respectively. As shown, the F-actins and cell nuclei of the cultured cells (HFF) together with the secreted type III collagen were clearly identified. Type I collagen was also found in the study (data not shown). However, the labeled type I collagen may come from the originally coated bovine collagen or that secreted by the cultured cells. These results indicated the cultured cells could secrete their own ECM during culture. On the contrary, the originally coated bovine type I collagen may degrade gradually. It was reported that human skin fibroblasts could secrete collagenase as two proenzyme forms. These enzymes play an essential role in the maintenance of the ECM during tissue development and remodeling (Proc. Natl. Acad. Sci. U.S.A. 1986;83:3756).

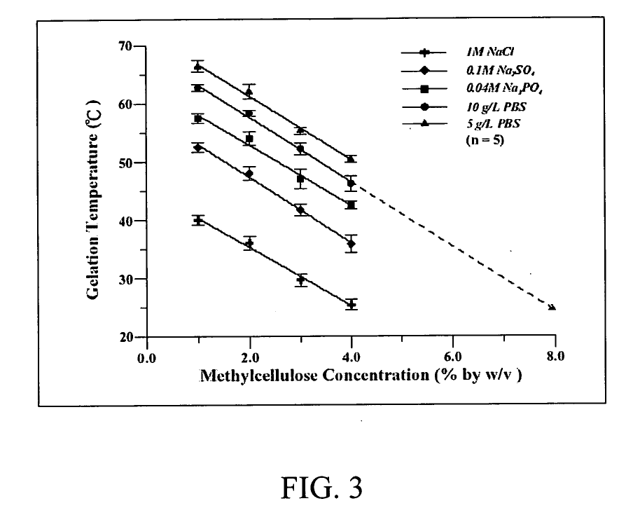

It was found that the concentration of MC in aqueous solution also played a significant role in its physical sol-gel behavior. As shown in FIG. 3, the gelation temperatures of aqueous MC solutions blended with distinct salts decreased approximately linearly with increasing the MC concentration. In the preparation of the aqueous MC solution, it was found that the solution was too viscous to be manipulated with when the MC concentration was greater than about 4% (by w/v). Therefore, no data were available when the concentration of MC was greater than this limit.

For the applications of cell culture, only those aqueous MC compositions that may form a gel (the MC hydrogel) at 37° C. were used to coat the TCPS dishes: 2% MC+1M NaCl; 2% MC+0.2M Na₂SO₄; 2% MC+0.2M Na₃PO₄ (FIG. 2); and 8% MC+10 g/L PBS. For the latter case, a 4% aqueous MC solution blended with 5 g/L PBS was used to coat the TCPS dish and subsequently dried in a laminar flow hood to remove 50% of its moisture content. Thus obtained MC hydrogel had a gelation temperature of about 25° C. (extrapolated from FIG. 3). As shown in FIG. 3, the gelation temperature of a 4% MC solution blended with PBS was significantly greater than 37° C. Additionally, as mentioned above, the aqueous MC solution was too viscous to be manipulated with when its concentration was greater than about 4%. It was observed that this specific aqueous MC solution (8% MC+10 g/L PBS) underwent a sol-gel reversible transition upon heating or cooling at approximately 25° C.

### Example No. 8 Stability of the Coated MC Hydrogel

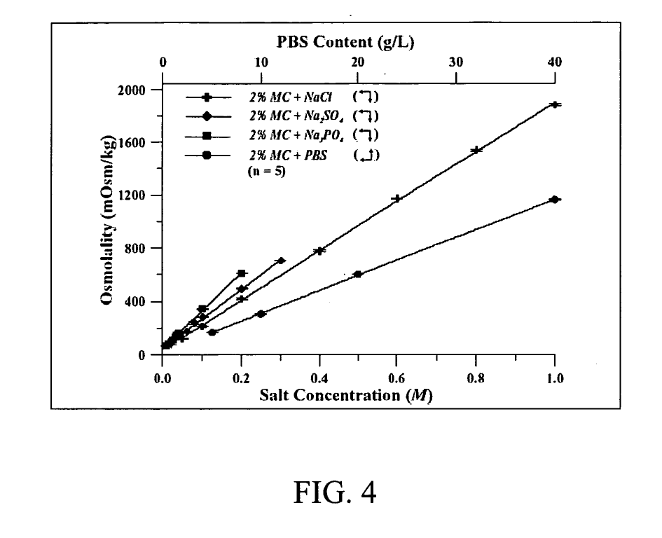

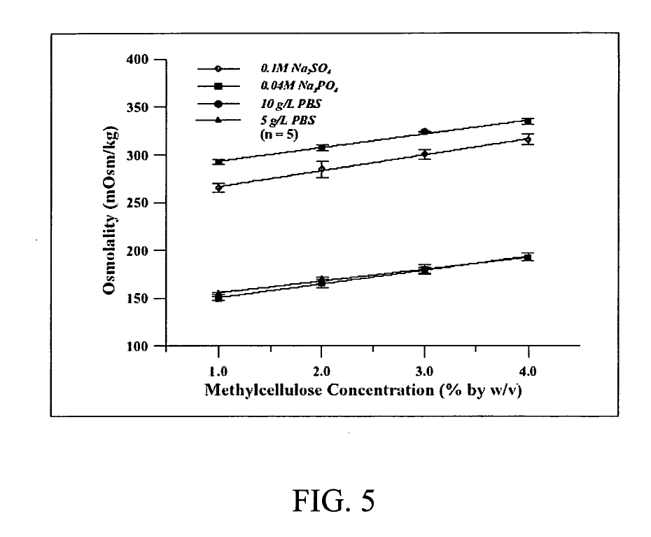

It is suggested that the MC hydrogels coated on TCPS dishes may be swelled and gradually disintegrated when loaded with the cell culture media due to the differences in osmotic pressure between the two. It was found that the osmolalities of aqueous MC solutions, used to prepare the MC hydrogels, increased nearly linearly with increasing the concentrations of the salt blended and MC (FIG. 4 and FIG. 5).

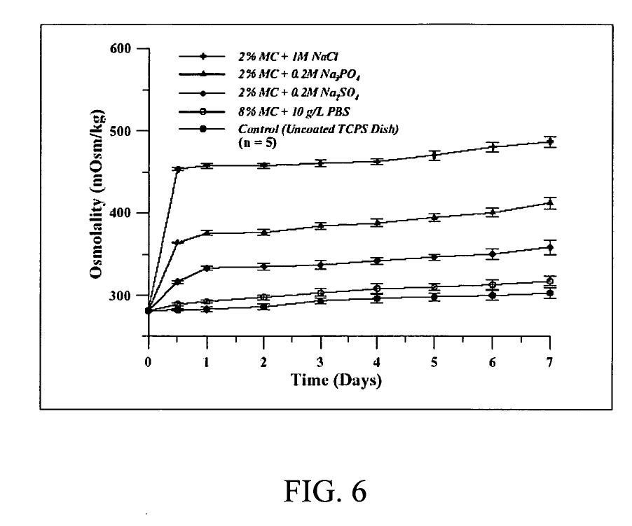

To evaluate the stability of the coated MC hydrogels, a PBS solution (10 g/L) with an osmolality of 280±10 mOsm/kg at 37° C., in simulating that of the cell culture media, was loaded on the coated TCPS dishes. The osmolality of the cell culture media is normally maintained at 290±30 mOsm/kg. An uncoated TCPS dish loaded with the same PBS solution was used as a control. Changes in osmolality of the loaded PBS solution with time were monitored by an osmometer. As compared to the uncoated control group, the osmolalities of the loaded PBS solutions increased significantly within 1 day (>325 mOsm/Kg) for the MC hydrogels blended with NaCl, Na₂SO₄, or Na₃PO₄ (p<0.05, FIG. 6). This observation might be attributed to the differences in osmolality between these MC hydrogels (>500 mOsm/kg, FIG. 4) and the originally loaded PBS solutions (about 280 mOsm/kg), and thus caused a significant amount of water from the loaded PBS solutions diffusing into the MC hydrogels. This leads to a significant increase in osmolality for the loaded PBS solutions together with a noticeable swelling and gradual disintegration of the MC hydrogels.

In contrast, the osmotic pressure of the PBS solution (10 g/L) loaded on the MC hydrogel blended with PBS (10 g/L) only increased slightly as compared to the uncoated control group (FIG. 6). Additionally, the MC hydrogel coated on the TCPS dish stayed intact throughout the entire course of the experiment. The aforementioned results indicated that the MC hydrogel blended with PBS (8% by w/v MC+10 g/L PBS) was more suitable for cell cultures than those blended with NaCl, Na₂SO₄, or Na₃PO₄, and thus was chosen for the study (the MC/PBS hydrogel).

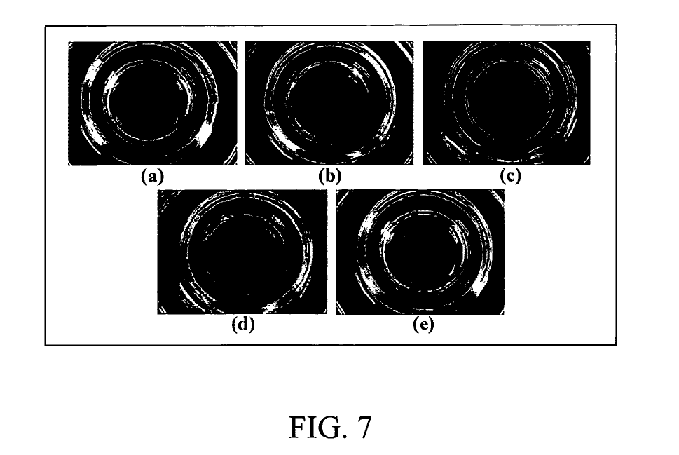

As shown in FIG. 7a, the MC/PBS hydrogel at 20° C. was a clear viscous solution. At 37° C., the clear solution starts to become opaque (FIG. 7b). The transition of sol-gel was continuous with time. At about 30 minutes later, a gel-network structure began to form (FIG. 7c). It was found that this hydrogel was thermoreversible. Back at 20° C., the opaque gel gradually became a clear viscous solution again (FIGS. 7d and 7e).

### Example No. 9 Cell Culture on the Surface of the MC Hydrogel

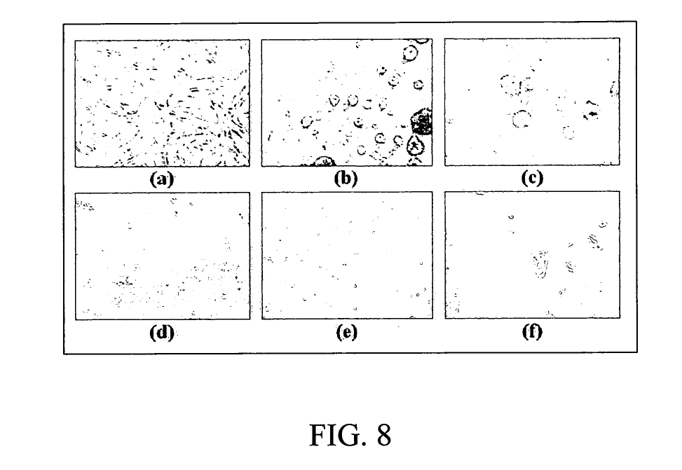

FIGS. 8a to 8f shows photomicrographs of cells (human foreskin fibroblasts, HFF) cultured on the surface of an uncoated TCPS dish (the control group) and those coated with the MC hydrogels blended with distinct slats for 1 day, respectively. As shown, the seeded cells attached very well on the surface of the uncoated TCPS dish (FIG. 8a). However, cells did not attach at all on the surfaces of the MC hydrogels blended with NaCl, Na₂SO₄, or Na₃PO₄ and mainly suspended in the culture media in the form of aggregates (FIGS. 8b-8d). In contrast, a few cells were found to attach on the surface of the MC/PBS hydrogel and the others remained to suspend in the culture media (FIGS. 8e and 8f).

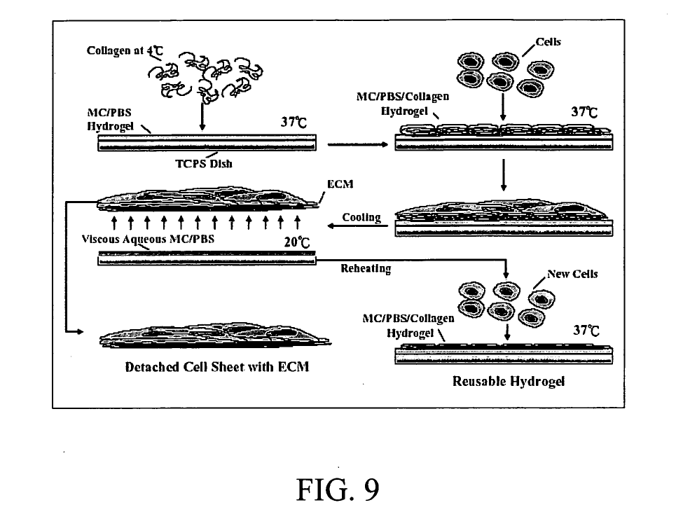

To improve cell attachments, a neutral aqueous bovine type I collagen at 4° C. was evenly spread on the TCPS dish coated with the MC/PBS hydrogel at 37° C. (FIG. 9). It was reported that under the influence of increasing temperature, collagen molecules self-assemble into a gel network. Thermal triggering of collagen gelation was demonstrated at a temperature as low as 20° C. and at a concentration as low as 0.1 mg/ml. Thus a thin layer of bovine type I collagen was formed on the surface of the MC/PBS hydrogel gradually (the MC/PBS/Collagen hydrogel, FIG. 9).

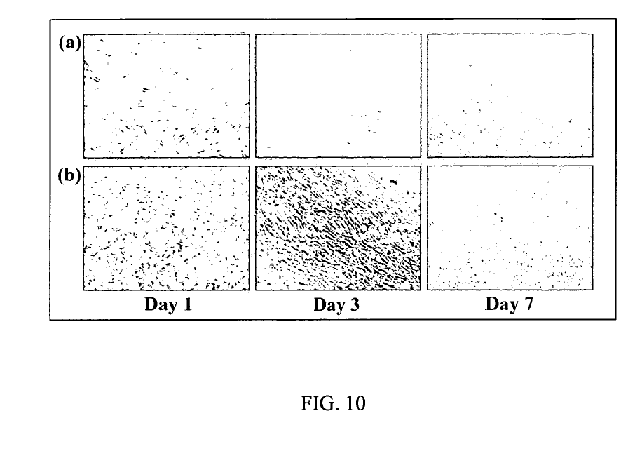

FIGS. 10a to 10i presents photomicrographs of cells cultured on an uncoated TCPS dish and that coated with the MC/PBS/Collagen hydrogel for 1, 3, and 7 days, respectively. Results of their relative-cell-activities of test-to-control evaluated by the MTT assay are shown in Table 2. As shown, after coating with the bovine type I collagen, cell attachments and proliferations were significantly improved as compared to those observed on the surface of the MC/PBS hydrogel (FIGS. 8e and 8f). The results obtained in the MTT assay demonstrated that the cells cultured on the surface of the MC/PBS/Collagen hydrogel had an even better activity than those cultured on the uncoated TCPS dish (p<0.05). Collagen is known to have the capacity to regulate cell behaviors such as adhesion, spreading, proliferation, and migration and thus has been used extensively to enhance cell-material interactions for both in vivo and in vitro applications.

TABLE 2
Results of the relative-cell-activities of test-to-control obtained in the
MTT assay for the cells cultured on an uncoated TCPS dish
(Uncoated Dish) and the TCPS dish coated with the
MC/PBS/Collagen hydrogel (Coated Dish)
for 1, 3, and 7 days, respectively (n = 5).

| Relative | | | |
|---|---|---|---|
| Cell Activity^{[a]} | Day 1 | Day 3 | Day 7 |
| Uncoated Dish | 100.0 ± 2.3% | 161.9 ± 9.4% | 203.0 ± 12.3% |
| Coated Dish | 159.1 ± 7.7% | 286.3 ± 13.5% | 339.9 ± 18.7% |

^{[a]}The cell activity of the cells cultured on the uncoated TCPS dish for 1 day was used as a control.

### Example No. 10 Detachment of Cell Sheets

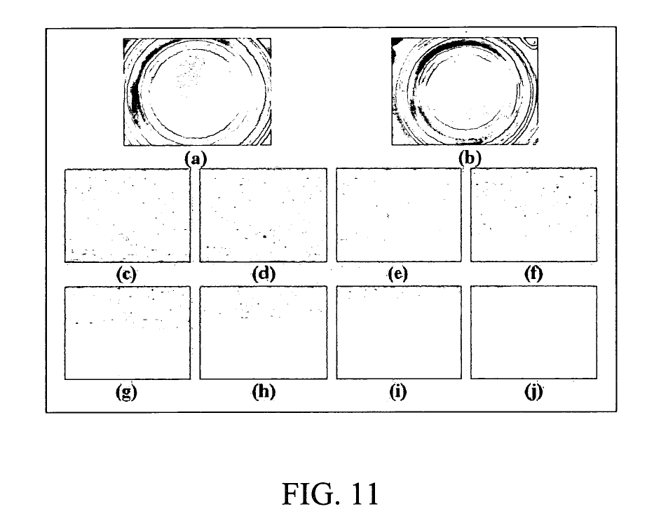

After cells reaching confluence, a continuous monolayer cell sheet formed on the surface of the MC/PBS/Collagen hydrogel (FIGS. 9 and 11a). When the grown cell sheet was placed outside of the incubator at 20° C., it detached gradually from the surface of the thermoreversible hydrogel spontaneously, in absence of any enzymes (e.g., trypsin/EDTA, FIGS. 9 and 11b-11j). It was observed that the grown cell sheet started to detach from its edge at about 2 minutes after cooling at 20° C. Detachment of the entire cell sheet was completed within 20 minutes (or within 10 minutes by shaking the TCPS dish gently with hand). With the same method, a large size of living cell sheet, cultured on a coated 100-mm petri dish, can be readily obtained in our lab and may be utilized in the applications of tissue reconstructions. FIG. 11 shows photographs of (a) a grown cell sheet on the TCPS dish coated with the MC/PBS/Collagen hydrogel and (b) its detaching cell sheet. Photomicrographs of the detaching cell sheet with time (c) to (O).

For most types of cells, and especially for a connective-tissue cell, the opportunities for anchorage and attachment depend on the surrounding matrix, which is usually made by the cell itself. It is known that fibroblasts are dispersed in connective tissue throughout the body, where they secrete an extracellular matrix (ECM) that is rich in type I and/or type III collagen (Molecular Biology of The Cell, 4^{th} ed., Garland Science, New York 2002, Ch. 22). The detached cell sheet was fixed and immunostained with anti-type I or type III collagen and subsequently co-stained with phalloidin for F-actins and propidium iodide for nuclei acids.

### Example No. 11 Applications of the Developed Technique

After harvesting the detached cell sheet, the remained viscous MC/PBS hydrogel can be reused subsequent to recoating a thin layer of type I collagen on its surface as described before (FIG. 9). Additionally, a multi-layer cell sheet can be obtained with one of the following two methods. For the first method, a double-layer cell sheet can be obtained by seeding new cells directly on top of the first grown cell sheet (without detaching it from the surface of the MC/PBS/Collagen hydrogel) and then culture until confluence (FIG. 10b). The same procedure can be repeated again to obtain a tri-layer cell sheet (FIG. 10c). The other method is to fold the detached cell sheet into multi layers and reculture it. The folded multi-layer cell sheet would then stick together between layers within 2 days and form an integrated multi-layer cell sheet (FIG. 10d).

Some aspects of the present invention provide a method of preparing a living cell sheet comprising: coating a thermoreversible hydrogel on a tissue culture dish, wherein said hydrogel comprises methylcellulose, and phosphate buffered saline; loading target living cells into said dish; incubating said dish for a predetermined duration; and removing said sheet from said dish. In one embodiment, the hydrogel further comprises collagen. In another embodiment, the hydrogel further comprises at least one growth factor. In another embodiment, the target living cells are mesenchymal stem cells and/or adult multipotent cells.

The aforementioned single-layer or multi-layer cell sheets may be used in the applications of tissue reconstructions or tissue regeneration. Cell sheet engineering is being developed as an alternative approach for tissue engineering. It may have the advantages of eliminating the use of biodegradable scaffolds and maintaining the cultured cell-cell and cell-ECM interactions.

In some aspects, the single-layer living cell sheet passes through a laser-assisted cell identification and separation process, wherein a laser light with a cell-specific frequency passes through all cells on the cell sheet in a rotating or programmed manner to identify distinct cells to be preserved (for example, the myocardial stem cells in adipose derived tissue cells). For those non-specific cells or unwanted cells, a laser light with cell destroying energy is emitted to kill those cells. Thereafter, only desired cell type from the single-layer living cell sheet is obtained for cell differentiation and cell regeneration in a recipient. In one embodiment, fluorescence-coded cells or fluorescence light may be used for identifying distinct cells to be preserved to improve the purity of the living cell sheet.

By substituting the tissue culture dish with a 3-dimensional scaffold support element, hydrogel or partially gelled hydrogel of the invention may be coated onto the support element, followed by loading the target living cells and incubation. In one embodiment, the 3-D scaffold support element is biodegradable or bioresorbable so that the cells-loaded support element serves as an implant for in situ tissue regeneration in a recipient. The biodegradable material for the scaffold support element may be selected from a group consisting of chitosan, collagen, elastin, gelatin, fibrin glue, biological sealant, and combination thereof. The biodegradable material for the scaffold support element may be selected from a group consisting of polylactic acid (PLA), polyglycolic acid (PGA), poly (D,L-lactide-co-glycolide), polycaprolactone, and co-polymers thereof. The biodegradable material for the scaffold support element may be selected from a group consisting of polyhydroxy acids, polyalkanoates, polyanhydrides, polyphosphazenes, polyetheresters, polyesteramides, polyesters, and polyorthoesters.

Some aspects of the present invention provides a method of preparing a 3-D living cell construct comprising: coating a thermoreversible hydrogel on a 3-D scaffold support element, wherein said hydrogel comprises methylcellulose, phosphate buffered saline, and collagen; loading target living cells onto said support element; incubating said support element for a predetermined duration. In one embodiment, the method further comprises a step of removing said construct from said support element.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

What is claimed is:

1. A method of preparing a living cell sheet comprising:
coating a thermoreversible hydrogel on a tissue culture dish, wherein said hydrogel comprises methylcellulose, and phosphate buffered saline;
loading target living cells into said dish;
incubating said dish for a predetermined duration; and
removing said sheet from said dish.

2. The method of claim 1, wherein the living cell sheet is a single-layer cell sheet.

3. The method of claim 1, wherein the living cell sheet is a multiple-layer cell sheet.

4. The method of claim 3, wherein the method further comprises a step, prior to the step of removing said sheet from said dish, of seeding new cells directly on top of a first grown cell sheet and culturing.

5. The method of claim 1, wherein said dish is incubated at a temperature higher than 20° C.

6. The method of claim 1, wherein said dish is incubated for at least one hour.

7. The method of claim 1, wherein said hydrogel further comprises at least one growth factor.

8. The method of claim 7, wherein the at least one growth factor is selected from a group consisting of vascular endothelial growth factor, basic fibroblast growth factor, acidic fibroblast growth factor, platelet derived growth factor, platelet derived angiogenesis factor, transforming growth factor-β, platelet derived epithelial growth factor, platelet derived wound healing factor, insulin-like growth factor, epidermal growth factor, hepatocytic growth factor, and combinations thereof.

9. The method of claim 1, further comprising a step of purifying living cells after the incubating step.

10. The method of claim 1, wherein said target living cells are mesenchymal stem cells.

11. The method of claim 1, wherein said target living cells are adult multipotent cells.

12. The method of claim 1, wherein said hydrogel further comprises collagen.

13. The method of claim 12, wherein the method further comprises a step, prior to the step of removing said sheet from said dish, of seeding new cells directly on top of a first grown cell sheet and culturing.

14. The method of claim 12, wherein said dish is incubated at a temperature higher than 20° C.

15. The method of claim 12, wherein said hydrogel further comprises at least one growth factor.

16. The method of claim 15, wherein the at least one growth factor is selected from a group consisting of vascular endothelial growth factor, basic fibroblast growth factor, acidic fibroblast growth factor, platelet derived growth factor, platelet derived angiogenesis factor, transforming growth factor-β, platelet derived epithelial growth factor, platelet derived wound healing factor, insulin-like growth factor, epidermal growth factor, hepatocytic growth factor, and combinations thereof.

17. The method of claim 1, wherein said target living cells are mesenchymal stem cells or adult multipotent cells.

18. A method of preparing a 3-D living cell construct comprising:
coating a thermoreversible hydrogel on a 3-D scaffold support element, wherein said hydrogel comprises methylcellulose, phosphate buffered saline, and collagen;
loading target living cells onto said support element;
incubating said support element for a predetermined duration.

19. The method of claim 18, further comprising a step of removing said construct from said support element.

20. The method of claim 18, wherein said support element is biodegradable.
